(19) United States
(12) Patent Application Publication
(10) Pub. No.: US 2016/0317155 A1
(43) Pub. Date: Nov. 3, 2016

(54) ATRIAL-APPENDAGE LIGATION TREATMENT TOOL AND ATRIAL-APPENDAGE LIGATION SYSTEM

(71) Applicant: OLYMPUS CORPORATION, Tokyo (JP)

(72) Inventors: Takehiro KIMURA, Tokyo (JP); Kazutoshi KUMAGAI, Tokyo (JP); Yoshiro OKAZAKI, Tokyo (JP); Naoya SUGIMOTO, Tokyo (JP); Hiroki HIBINO, Tokyo (JP); Yoshiyuki KUMADA, Tokyo (JP); Hiroshi FUKUDA, Tokyo (JP)

(73) Assignee: OLYMPUS CORPORATION, Tokyo (JP)

(21) Appl. No.: 15/208,759

(22) Filed: Jul. 13, 2016

Related U.S. Application Data
(63) Continuation of application No. PCT/JP2015/053953, filed on Feb. 13, 2015.

(30) Foreign Application Priority Data
Mar. 31, 2014 (JP) 2014-074157

(51) Int. Cl. A61B 17/12; A61B 17/29; A61B 17/128

(52) U.S. Cl. CPC A61B 17/12013; A61B 17/1285; A61B 17/29; A61B 2017/00243

(57) ABSTRACT

Even if atrial-appendage tissue is damaged due to an excessive force unintentionally exerted during ligation of the atrial appendage, the effect of the damage is minimized. Provided is an atrial-appendage ligation treatment tool that is used, together with an atrial-appendage ligation tool having a ligation part, to perform atrial-appendage ligation treatment. The atrial-appendage ligation treatment tool includes: a compressing portion that is introduced, separately from the ligation part, to a cardiac sac through a sheath penetrated through a pericardium and that compresses the atrial appendage from outside to occlude an internal space therein; and a shaft that is disposed in the sheath in an inserted manner and that supports, at a distal end thereof, the compressing portion.

### CROSS-REFERENCE TO RELATED APPLICATIONS

This is a continuation of International Application PCT/JP2015/053953 which is hereby incorporated by reference herein in its entirety.

This application is based on Japanese Patent Application No. 2014-074157, the contents of which are incorporated herein by reference.

### TECHNICAL FIELD

The present invention relates to an atrial-appendage ligation treatment tool and an atrial-appendage ligation system.

### BACKGROUND ART

In recent years, the number of patients with atrial fibrillation, which is one type of arrhythmia, has exhibited an increasing tendency. It is considered that brain infarction caused by atrial fibrillation occurs when a blood vessel in the brain is blocked by a blood clot formed in the heart (mainly, in the left atrial appendage). The most-frequent embolic source for the cardiogenic cerebral embolism is a blood clot in the left atrial appendage due to atrial fibrillation.

A common treatment recommended for prevention of cardiogenic cerebral embolisms is blood anticoagulant therapy with warfarin potassium; however, the administration of warfarin potassium is difficult to manage, and warfarin potassium has a risk of bleeding complications. As a substitute for this, a method of preventing embolisms by occluding the left atrial appendage has been developed (for example, Watchman, Boston Scientific). This is a jellyfish-shaped device for occluding the left atrial appendage, like a transvascular catheter.

On the other hand, a treatment tool for ligating the atrial appendage from outside the heart, without using an anticoagulant drug and without entering a blood vessel, is known (for example, see PTL 1). This is a treatment tool in which forceps and a ligation loop are inserted into the cardiac sac from outside the body, the ligation loop is looped around the atrial appendage while an end portion of the atrial appendage is being grasped and pulled by using the grasping forceps, and then the ligation loop is tightened, thus ligating the atrial appendage.

This treatment tool is provided with a sleeve that accommodates the ligation loop in a concave portion thereof, so as to facilitate looping of the ligation loop around the atrial appendage by keeping the ligation loop spread by using the sleeve.

### CITATION LIST

### Patent Literature

### {PTL 1} US Patent Application, Publication No. 2008/0294175

### SUMMARY OF INVENTION

### Solution to Problem

According to one aspect, the present invention provides an atrial-appendage ligation treatment tool that is used, together with an atrial-appendage ligation tool having a ligation part, to perform atrial-appendage ligation treatment, the atrial-appendage ligation treatment tool including: a compressing portion that is introduced, separately from the ligation part, to a cardiac sac through a sheath penetrated through a pericardium and that compresses an atrial appendage from outside to occlude an internal space therein; and a shaft that is disposed in the sheath in an inserted manner and that supports, at a distal end thereof, the compressing portion.

### BRIEF DESCRIPTION OF DRAWINGS

FIG. 1 is a perspective view showing an atrial-appendage ligation treatment tool according to one embodiment of the present invention.

FIG. 2 is a cross-sectional view showing a state in which the atrial-appendage ligation treatment tool shown in FIG. 1 is accommodated in a sheath.

FIG. 3 is a perspective view showing a state in which an atrial appendage is sandwiched between two contact parts of a pressing portion of the atrial-appendage ligation treatment tool shown in FIG. 1, with the contact parts spread apart.

FIG. 4 is a perspective view showing a state in which the two contact parts of the atrial-appendage ligation treatment tool shown in FIG. 1 are closed to compress the atrial appendage.

FIG. 5 is a perspective view for explaining a state in which a ligation loop is looped around the atrial appendage, from the state shown in FIG. 3.

FIG. 6 is a perspective view showing a state in which the ligation loop looped around the atrial appendage in FIG. 5 is brought close to the pressing portion of the atrial-appendage ligation treatment tool.

FIG. 7 is a perspective view showing a state in which the two contact parts of the atrial-appendage ligation treatment tool shown in FIG. 1 are closed to occlude an internal space in the atrial appendage, in the middle of treatment shown in FIG. 5.

FIG. 8 is a front view showing a pressing portion in which the contact parts are made to attract each other by using a magnet, according to a first modification of the atrial-appendage ligation treatment tool shown in FIG. 1.

FIG. 9A is a perspective view showing, in another method of bending a shaft, a state where a hollow shaft is rectified by a high-stiffness rod, according to a second modification of the atrial-appendage ligation treatment tool shown in FIG. 1.

FIG. 9B is a perspective view showing, in the method of bending the shaft, a state where the rod is withdrawn, thus letting the hollow shaft bend due to a pre-bent section, according to the second modification of the atrial-appendage ligation treatment tool shown in FIG. 1.

FIG. 10 is a perspective view showing a pressing portion in which the pressing portion is bent by using a joint, according to a third modification of the atrial-appendage ligation treatment tool shown in FIG. 1.

FIG. 11A is a perspective view showing a pressing portion in which bending of the pressing portion with respect to the shaft and opening/closing of the two contact parts are performed by using joints and a state in which the pressing portion is linearly extended, according to a fourth modification of the atrial-appendage ligation treatment tool shown in FIG. 1.

FIG. 11B is a perspective view showing the pressing portion in which bending of the pressing portion with respect to the shaft and opening/closing of the two contact parts are performed by using the joints and a state in which only the contact parts are bent, according to the fourth modification of the atrial-appendage ligation treatment tool shown in FIG. 1.

FIG. 11C is a perspective view showing the pressing portion in which bending of the pressing portion with respect to the shaft and opening/closing of the two contact parts are performed by using the joints and a state in which the contact parts are opened and closed, according to the fourth modification of the atrial-appendage ligation treatment tool shown in FIG. 1.

FIG. 12A is a perspective view showing the overall configuration of an atrial-appendage ligation treatment tool that integrally holds a ligation loop, according to a fifth modification of the atrial-appendage ligation treatment tool shown in FIG. 1.

FIG. 12B is a cross-sectional view showing, in an enlarged manner, a holding part in the atrial-appendage ligation treatment tool that integrally holds the ligation loop, according to the fifth modification of the atrial-appendage ligation treatment tool shown in FIG. 1.

FIG. 13A is a view showing a state in which a loop is looped around the atrial appendage in an atrial-appendage ligation system according to the embodiment of the present invention.

FIG. 13B is a view showing a state in which grasping forceps are moved along the shaft to be brought close to the atrial appendage, in the atrial-appendage ligation system according to the embodiment of the present invention.

FIG. 13C is a view showing a state in which the atrial appendage is grasped and pulled by the grasping forceps, in the atrial-appendage ligation system according to the embodiment of the present invention.

FIG. 13D is a view showing a state in which the loop is tightened to occlude the internal space in the atrial appendage, in the atrial-appendage ligation system according to the embodiment of the present invention.

FIG. 14A is a view showing a state of a manipulating portion when the space between the contact parts is widened to the maximum, according to a sixth modification of the atrial-appendage ligation treatment tool shown in FIG. 1.

FIG. 14B is a view showing a state of the pressing portion when the manipulating portion is in the state shown in FIG. 14A, according to the sixth modification of the atrial-appendage ligation treatment tool shown in FIG. 1.

FIG. 14C is a view showing a state of the manipulating portion when the space between the contact parts is narrowed halfway, according to the sixth modification of the atrial-appendage ligation treatment tool shown in FIG. 1.

FIG. 14D is a view showing a state of the pressing portion when the manipulating portion is in the state shown in FIG. 14C, according to the sixth modification of the atrial-appendage ligation treatment tool shown in FIG. 1.

FIG. 14E is a view showing a state of the manipulating portion when the space between the contact parts is narrowed to the minimum, according to the sixth modification of the atrial-appendage ligation treatment tool shown in FIG. 1.

FIG. 14F is a view showing a state of the pressing portion when the manipulating portion is in the state shown in FIG. 14E, according to the sixth modification of the atrial-appendage ligation treatment tool shown in FIG. 1.

FIG. 15A is a view showing a state of the manipulating portion when the space between the contact parts is widened to the maximum, according to a modification of the atrial-appendage ligation treatment tool shown in FIGS. 14A to 14F.

FIG. 15B is a view showing a state of the pressing portion when the manipulating portion is in the state shown in FIG. 15A, according to the modification of the atrial-appendage ligation treatment tool shown in FIGS. 14A to 14F.

FIG. 15C is a view showing a state of the manipulating portion when the space between the contact parts is narrowed halfway, according to the modification of the atrial-appendage ligation treatment tool shown in FIGS. 14A to 14F.

FIG. 15D is a view showing a state of the pressing portion when the manipulating portion is in the state shown in FIG. 15C, according to the modification of the atrial-appendage ligation treatment tool shown in FIGS. 14A to 14F.

FIG. 15E is a view showing a state of the manipulating portion when the space between the contact parts is narrowed to the minimum, according to the modification of the atrial-appendage ligation treatment tool shown in FIGS. 14A to 14F.

FIG. 15F is a view showing a state of the pressing portion when the manipulating portion is in the state shown in FIG. 15E, according to the modification of the atrial-appendage ligation treatment tool shown in FIGS. 14A to 14F.

FIG. 16A is a cross-sectional view of a manipulating portion according to a modification of the atrial-appendage ligation treatment tool shown in FIGS. 14A to 14F.

FIG. 16B is a front view of a base end of the manipulating portion according to the modification of the atrial-appendage ligation treatment tool shown in FIGS. 14A to 14F.

FIG. 17A is a view showing a state of the manipulating portion when the space between the contact parts is widened to the maximum, for explaining a movement in the modification shown in FIGS. 16A and 16B.

FIG. 17B is a view showing a state of the pressing portion when the manipulating portion is in the state shown in FIG. 17A, for explaining the movement in the modification shown in FIGS. 16A and 16B.

FIG. 17C is a view showing a state of the manipulating portion when the space between the contact parts is narrowed halfway, for explaining the movement in the modification shown in FIGS. 16A and 16B.

FIG. 17D is a view showing a state of the pressing portion when the manipulating portion is in the state shown in FIG. 17C, for explaining the movement in the modification shown in FIGS. 16A and 16B.

FIG. 17E is a view showing a state of the manipulating portion when the space between the contact parts is narrowed to the minimum, for explaining the movement in the modification shown in FIGS. 16A and 16B.

FIG. 17F is a view showing a state of the pressing portion when the manipulating portion is in the state shown in FIG. 17E, for explaining the movement in the modification shown in FIGS. 16A and 16B.

FIG. 18 is a partially cutaway perspective view showing the modification shown in FIGS. 16A and 16B.

FIG. 19A is a perspective view showing a state in which a loop formed of a wire is open, according to a seventh modification of the atrial-appendage ligation treatment tool shown in FIG. 1.

FIG. 19B is a perspective view showing a state in which the loop is closed, according to the seventh modification of the atrial-appendage ligation treatment tool shown in FIG. 1.

FIG. 20A is a perspective view showing a state in which a loop formed of wires is open, according to an eighth modification of the atrial-appendage ligation treatment tool shown in FIG. 1.

FIG. 20B is a perspective view showing a state in which the loop is closed, according to the eighth modification of the atrial-appendage ligation treatment tool shown in FIG. 1.

FIG. 21A is a perspective view showing a state in which a loop formed of wires is open to the maximum, according to a ninth modification of the atrial-appendage ligation treatment tool shown in FIG. 1.

FIG. 21B is a perspective view showing a state in which the loop is narrowed halfway, according to the ninth modification of the atrial-appendage ligation treatment tool shown in FIG. 1.

FIG. 21C is a perspective view showing a state in which the loop is narrowed to the minimum, according to the ninth modification of the atrial-appendage ligation treatment tool shown in FIG. 1.

### DESCRIPTION OF EMBODIMENT

An atrial-appendage ligation treatment tool 1 according to one embodiment of the present invention will be described below with reference to the drawings.

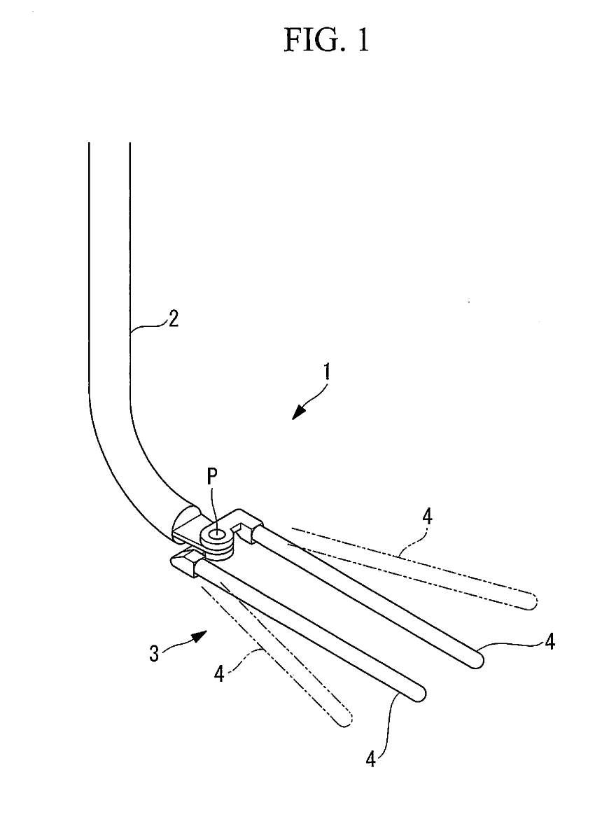

As shown in FIG. 1, the atrial-appendage ligation treatment tool 1 of this embodiment is provided with an elongated shaft 2 that is formed of an elastic material and that has a pre-bent section whose distal end is bent at about 90 degrees, and a pressing portion (compressing portion) 3 that is disposed at the distal end of the shaft 2. In this embodiment, the shaft 2 itself forms a biasing part for exerting a bias in the direction in which it is bent due to the pre-bent section.

The pressing portion 3 is formed into a two-pronged shape having two straight-rod-like contact parts 4 that are disposed in parallel, with a predetermined space therebetween. A curvature plane of the shaft 2 produced by the pre-bent section is approximately perpendicular to a plane in which the two contact parts 4 are disposed.

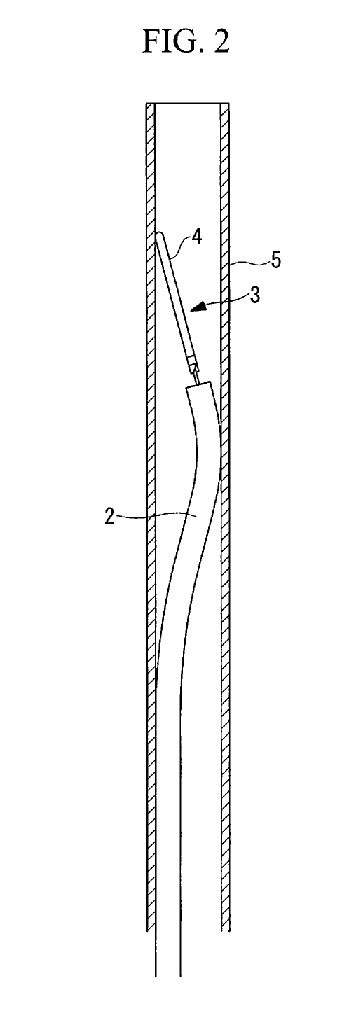

Although the shaft 2 can be bent along the shape of a sheath 5, the shaft 2 has stiffness so as to be able to transfer, in the longitudinal direction, a pressing force applied to a base end of the sheath 5. Furthermore, the pre-bent section of the shaft 2 is linearly extended by being inserted into the sheath 5, as shown in FIG. 2, so that the pressing portion 3 can be disposed almost at an extension of the shaft 2 in the longitudinal direction so as to be approximately aligned with the shaft 2.

As shown in FIG. 1, the contact parts 4 are each formed to be circular in cross section, and the distal ends of the contact parts 4 are rounded so as not to stick in tissue even when pressed against the tissue.

Furthermore, the two contact parts 4 are each provided so as to be rotatable about the shaft of a joint P, thereby allowing the space therebetween to be changed (the two contact parts 4 to be opened and closed).

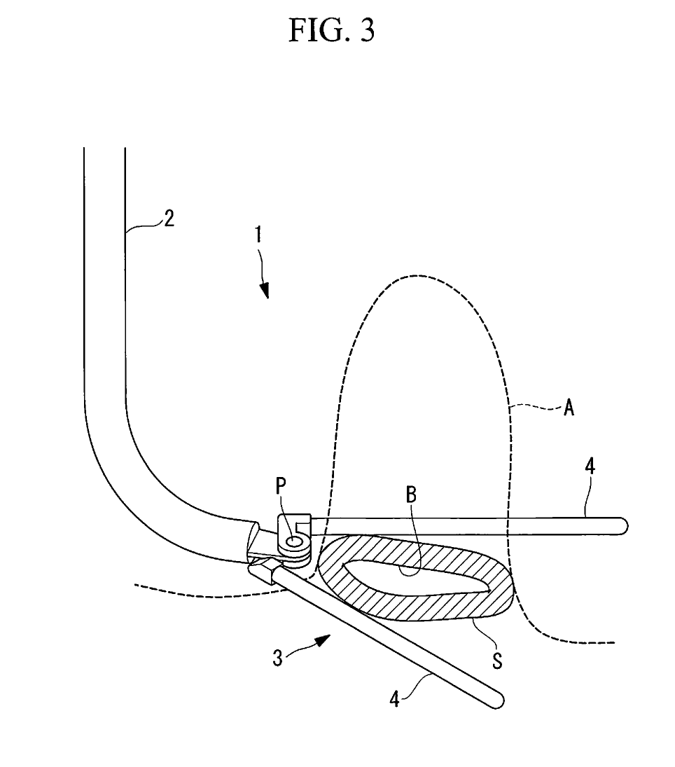

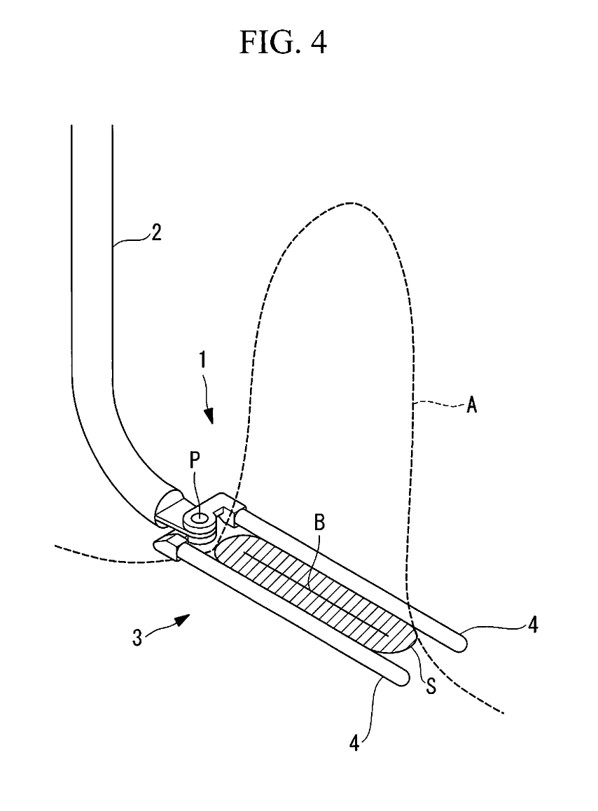

The contact parts 4 can be rotated by performing a pulling manipulation on a wire (not shown) that extends through the inside of the shaft 2 in the longitudinal direction from a base end thereof. Then, as shown in FIG. 3, when the space between the contact parts 4 is widened, the space is wider than the thickness dimension of the atrial appendage A in the normal state; and, as shown in FIG. 4, when the space therebetween is narrowed, the contact parts 4 are brought close to each other to have a space dimension such that the atrial appendage A can be compressed in the thickness direction, thus occluding an internal space B therein. In the figure, reference sign S denotes a cross section of the atrial appendage A at the position of the contact parts 4.

The procedure for ligating the atrial appendage A by using the thus-configured atrial-appendage ligation treatment tool 1 of this embodiment will be described below.

In order to ligate the atrial appendage A by using the atrial-appendage ligation treatment tool 1 of this embodiment, first, in a state in which a distal-end opening of the sheath 5 is disposed in the cardiac sac after being made to penetrate through body-surface tissue and the pericardium from a lower portion of the ensiform cartilage, the space between the contact parts 4 of the pressing portion 3 is narrowed to the minimum, and the atrial-appendage ligation treatment tool 1, with the pre-bent section of the shaft 2 being extended approximately linearly, is inserted into the sheath 5 and is made to advance toward the inside of the cardiac sac.

At this time, with the space between the contact parts 4 being narrowed to reduce the maximum breadth, the pressing portion 3 is approximately aligned with the shaft 2, thus allowing the pressing portion 3 to smoothly advance in the sheath 5. Then, when the pressing portion 3 comes out from the distal-end opening of the sheath 5 to the inside of the cardiac sac, the pre-bent section of the shaft 2 that has been restricted is released, thus bending, as shown in FIG. 1, and the pressing portion 3 is pointed in a direction intersecting the longitudinal direction of the shaft 2.

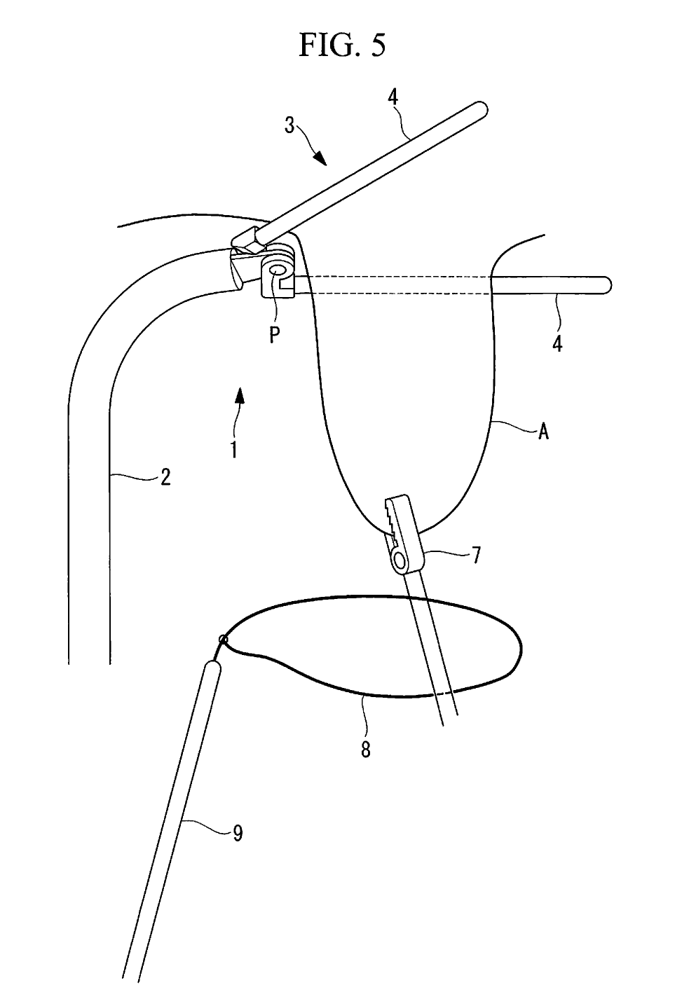

In this state, the space between the contact parts 4 of the pressing portion 3 is widened, as shown in FIG. 3, and, while performing observation with an endoscope that is separately inserted into the cardiac sac, the shaft 2 is manipulated outside the body near the base end of the sheath 5 to bring the pressing portion 3 close to the atrial appendage A from a distal end of the atrial appendage A, and the atrial appendage A is inserted between the two contact parts 4 of the pressing portion 3, as shown in FIG. 5.

Specifically, the shaft 2 is disposed at the right side of the left atrial appendage A (at the left side when viewed from the front). If observation with the endoscope is difficult, the shaft 2 may be moved to a location other than the right side of the left atrial appendage A, for example, to a location thereabove. Because the atrial appendage A is a sac-like tissue projecting, like an ear, from the outer surface of the heart and has flexibility, one of the contact parts 4, with the space therebetween being widened, is inserted, so as to be slipped behind the atrial appendage A, thereby making it possible to dispose the contact parts 4 at a position where the atrial appendage A is sandwiched between the contact parts 4 in the thickness direction.

Then, as shown in FIG. 5, the pressing portion 3 is made to advance while the distal end of the atrial appendage A is being pulled by grasping forceps 7 that are introduced through the sheath 5, thereby making it possible to press the contact parts 4 of the pressing portion 3 against the outer wall of the left atrium and to stretch the atrial appendage A.

In this state, a shaft (hereinafter, referred to as loop shaft) 9 of a ligation loop (ligation part) 8 that is brought close to the atrial appendage A from an outer side of the grasping forceps 7 and the grasping forceps 7 are simultaneously manipulated to loop the ligation loop 8 around the atrial appendage A.

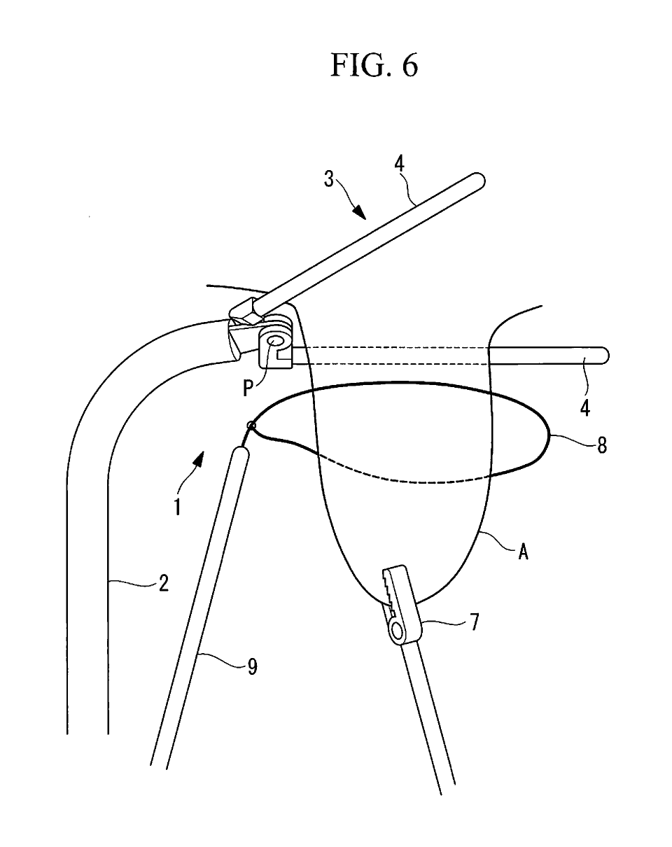

Specifically, with the end portion of the atrial appendage A being grasped by the grasping forceps 7, the loop shaft 9 is pushed at the same time as the grasping forceps 7 are pulled, and the grasping forceps 7 grasping the atrial appendage A are moved so as to be pulled out from the inside of the ligation loop 8, thereby making it possible to insert the atrial appendage A into the ligation loop 8 and to easily loop the ligation loop 8 around the atrial appendage A, as shown in FIG. 6.

Then, when the ligation loop 8 is positioned at the base portion of the atrial appendage A, which is stretched and exposed by being pulled by the grasping forceps 7 while the pressing portion 3 is being pressed against the outer wall of the left atrium, one end of the ligation loop 8 is pulled out from a base end of the loop shaft 9 while the loop shaft 9 is being pushed, thereby making it possible to tighten the ligation loop 8 to ligate the atrial appendage A.

In these series of movements, i.e., the movement of grasping the distal end of the atrial appendage A with the grasping forceps 7, the movement of pulling and stretching the atrial appendage A with the grasping forceps 7, and the movement of ligating the base of the atrial appendage A with the ligation loop 8, an operator exerts or determines a grasping force produced by the grasping forceps 7, a force stretching the atrial appendage A and the direction in which the atrial appendage A is stretched, and a pulling force for tightening the ligation loop 8; however, if an excessive force is unintentionally exerted, the atrial appendage A may be damaged in some cases.

Note that, although the ligation loop is used as the ligation part in this embodiment, instead of using a loop for ligation, it is possible to use a stapler (not shown), energy of ultrasonic waves or high-frequency waves (not shown), or a ligation part formed of a combination thereof.

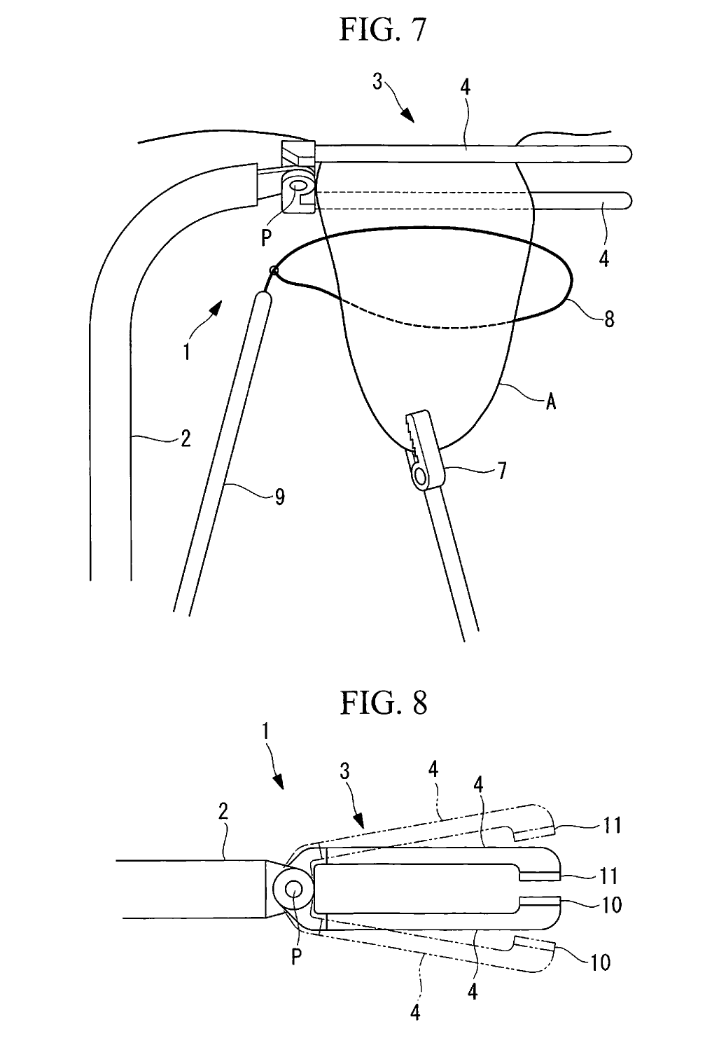

In such cases, as shown in FIG. 7, the two contact parts 4, which constitute the pressing portion 3 of the atrial-appendage ligation treatment tool 1 of this embodiment, are rotated in directions causing them to approach each other, thereby sandwiching the atrial appendage A between the two contact parts 4 and occluding the internal space B in the atrial appendage A, as shown in FIG. 4. Accordingly, the blood flow passing through the occluded portion is stemmed.

When a grasped portion of the atrial appendage A grasped by the grasping forceps 7 or a ligated portion thereof ligated by the ligation loop 8 is damaged, a portion thereof that is closer to the base than the damaged portion is can be compressed by the pressing portion 3 of the atrial-appendage ligation treatment tool 1, to temporarily suppress the blood flow into the damaged portion; therefore, there is an advantage that the effect of damage to the tissue of the atrial appendage A can be minimized. With the blood flow into the damaged portion being temporarily suppressed by the pressing portion 3, it is possible to subject the portion that is closer to the base than the damaged portion is to permanent treatment, such as ligation using the ligation loop 8 or thoracotomy and suturing.

In this way, according to the atrial-appendage ligation treatment tool 1 of this embodiment, the space between the two contact parts 4, which constitute the pressing portion 3, is narrowed, thereby allowing smooth passage in the sheath 5, and, after being introduced to the cardiac sac from the distal end of the sheath 5, the space between the two contact parts 4 is widened, thereby making it possible to facilitate insertion of the atrial appendage A therebetween. Furthermore, in the event of any damage being caused to the atrial appendage A, the space between the two contact parts 4 is narrowed to compress the atrial appendage A in the thickness direction, thus making it possible to stop a blood flow into the atrial appendage A.

Note that, as a mechanism for opening and closing the two contact parts 4, it is possible to adopt a mechanism for opening and closing the two contact parts 4 by using a wire that is introduced along the shaft 2, as in normal grasping forceps, or a mechanism in which a distal end portion 10 of one of the two contact parts 4 is formed of a magnetic material, and the other can be magnetized by an electromagnet 11, as shown in FIG. 8. After the atrial appendage A is inserted therebetween while the electromagnet 11 has not yet been magnetized, the electromagnet 11 is magnetized, thereby bringing the two contact parts 4 close to each other due to the magnetic force, and thus the atrial appendage A can be firmly sandwiched in the thickness direction, thus occluding the internal space therein.

Note that the electromagnet 11 may be provided on both of the two contact parts 4. Furthermore, in a case in which the two contact parts 4 are introduced through separate shafts 2, permanent magnets may be adopted instead of the electromagnets 11.

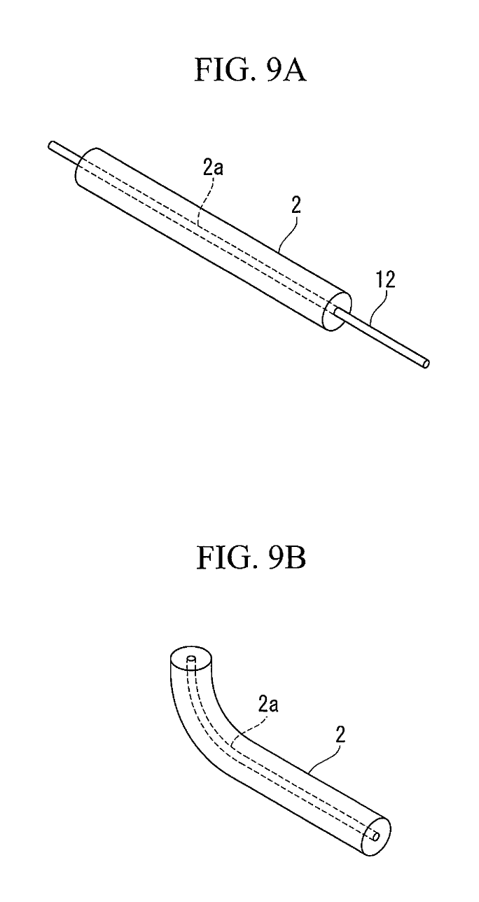

Furthermore, in this embodiment, although the pre-bent section of the shaft 2 is rectified by the sheath 5, instead of this, it is also possible to removably insert a straight rod 12 that has higher stiffness than the shaft 2 into a through-hole 2a penetrating the shaft 2 in the longitudinal direction thereof, thus rectifying the shaft 2, as shown in FIG. 9A, and to withdraw the rod 12 from the through-hole 2a, thereby letting the shaft 2 bend due to the pre-bent section thereof, as shown in FIG. 9B. Accordingly, compared with the case in which the shaft 2 is rectified by using the sheath 5, the friction force with respect to the sheath 5 is reduced, thus making it possible to improve the ease of introducing and withdrawing the shaft 2 to and from the cardiac sac.

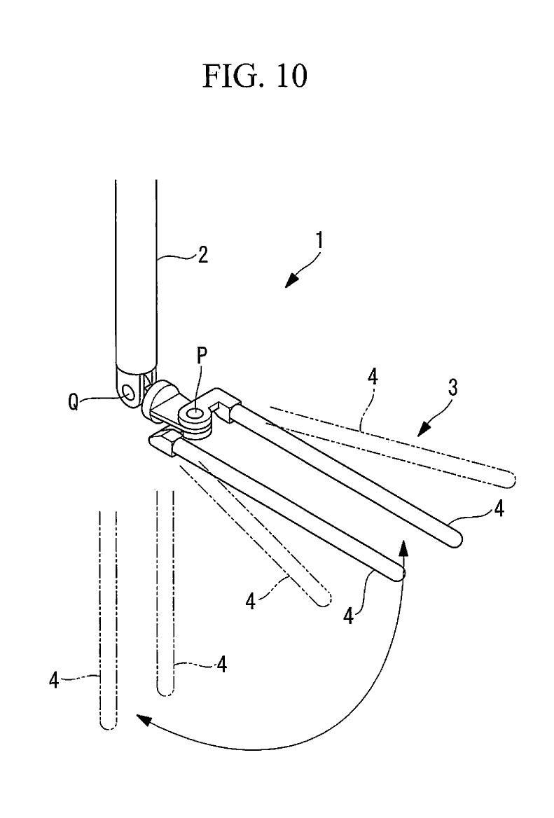

Furthermore, in this embodiment, the shaft 2, which is formed of an elastic material and has the pre-bent section, allows the pressing portion 3 to be disposed in the cardiac sac in a direction intersecting the longitudinal direction of the shaft 2. Instead of this, as shown in FIG. 10, it is possible to provide a joint Q between the shaft 2 and the pressing portion 3 and to exert a bias so as to rotate the joint Q by using a biasing part (not shown), such as a spring. At this time, the direction of rotation of the joint Q is along a plane perpendicular to a plane that includes the two contact parts 4.

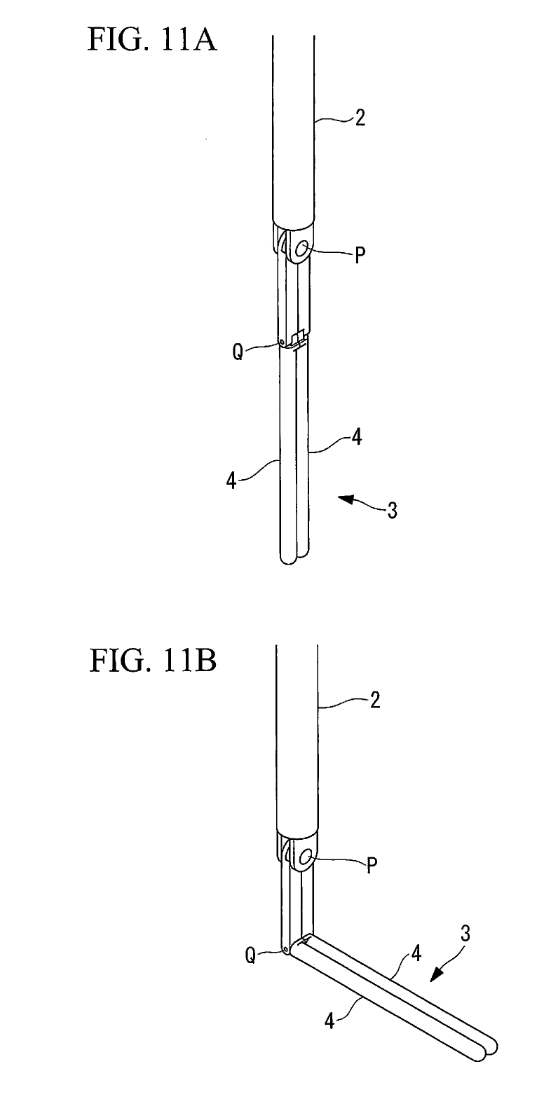

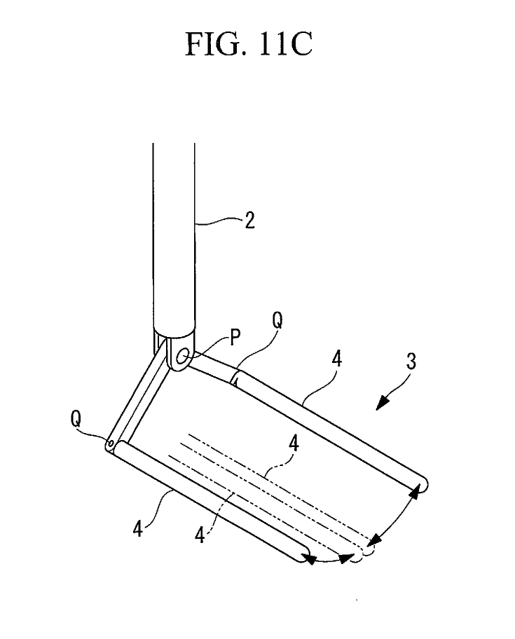

In order to change the angle formed by the shaft 2 and the pressing portion 3 by using the joint Q, as shown in FIG. 10, it is possible to provide the joint Q, which allows the entire pressing portion 3 to rotate with respect to the shaft 2, at a position that is closer to the base end than the joint P, which allows the two contact parts 4 of the pressing portion 3 to be opened and closed, is; alternatively, as shown in FIGS. 11A to 11C, it is possible to provide the joint P, which allows the two contact parts 4 to be opened and closed, at a position that is closer to the base end than the joint Q, which allows the contact parts 4 to rotate with respect to the shaft 2, is.

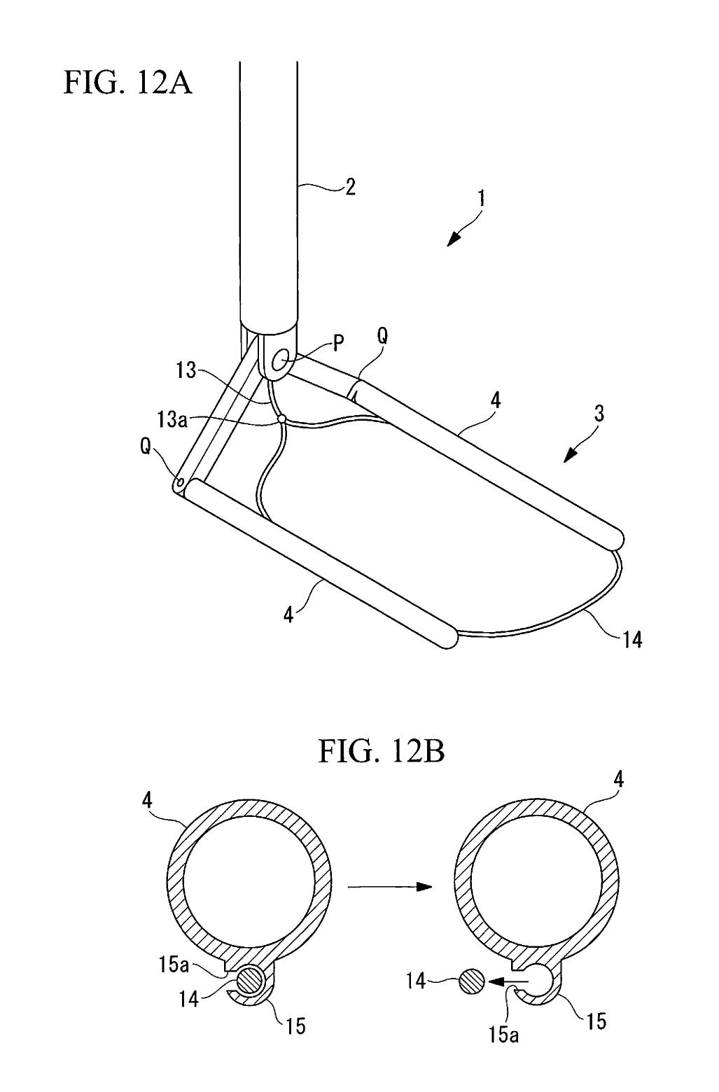

Furthermore, in this embodiment, a description has been given of, as an example, the atrial-appendage ligation treatment tool 1 having the compressing portion for compressing the atrial appendage in the thickness direction by using the two contact parts 4, which are opened and closed; however, instead of this, as shown in FIG. 12A, a loop-like wire 13 may be adopted as the compressing portion.

In the example shown in FIG. 12A, the shaft 2 of the atrial-appendage ligation treatment tool 1 is formed to be hollow, the wire 13 is penetrated therethrough, and a loop 14 is formed by a knot 13a at a position close to the distal end of the shaft 2. In this example, the contact parts 4 are provided with holding parts 15 that each hold part of the loop 14.

As shown in FIG. 12B, the holding parts 15 are each formed into a circular shape through which the wire 13, which forms the loop 14, passes, and are provided with slits 15a each having a width dimension slightly less than the outer-diameter dimension of the wire 13. Accordingly, in a state in which the loop 14 is not tightened, the holding parts 15 hold the loop 14, and the space between the contact parts 4 is widened, thereby opening the loop 14 and making it easy to insert the atrial appendage A therein. Furthermore, when the loop 14 is tightened to compress the atrial appendage A, the loop 14 is released, due to the tension thereof, from retention by the holding parts 15, which are provided with slits 15a.

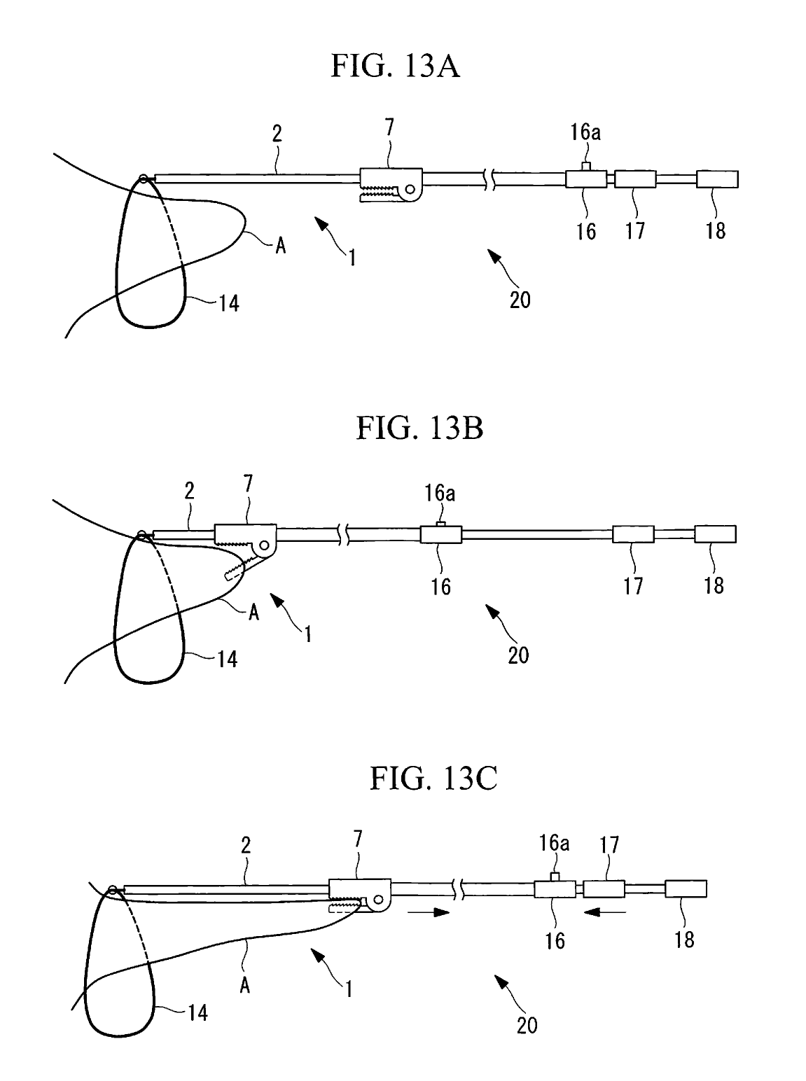

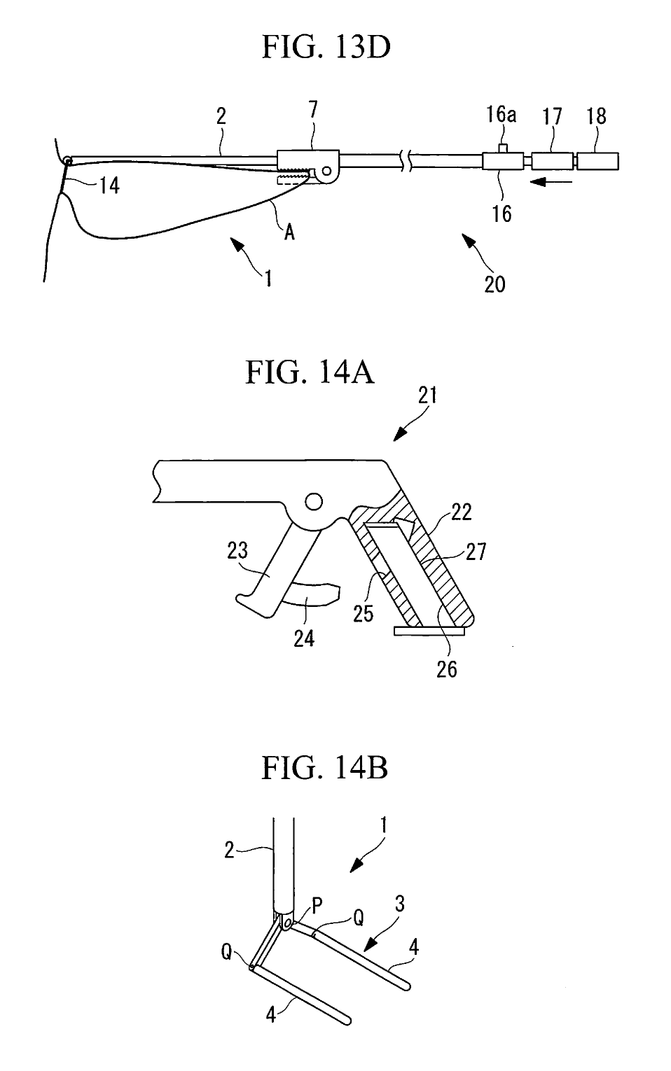

Furthermore, as shown in FIGS. 13A to 13D, it is possible to adopt an atrial-appendage ligation system 20 in which the grasping forceps 7 are attached, in such a manner as to be relatively movable in the longitudinal direction, along the shaft 2 of the atrial-appendage ligation treatment tool 1. Reference sign 16 denotes a manipulating portion for the grasping forceps 7; the manipulating portion is gripped to move the grasping forceps 7 back and forth, and the grasping forceps 7 can be opened and closed by using a push button 16a. Reference sign 17 denotes a handle for moving the loop 14 back and forth, and reference sign 18 denotes a handle for tightening the loop 14.

Specifically, as shown in FIG. 13A, with the loop 14 being open, the loop 14, which is provided at the distal end of the shaft 2 of the atrial-appendage ligation treatment tool 1, is looped around the atrial appendage A. As shown in FIG. 13B, the manipulating portion 16 is manipulated, and the push button 16a is pushed to open the grasping forceps 7 and to make the grasping forceps 7 advance along the shaft 2, thereby bringing them close to the distal end of the atrial appendage A.

Then, as shown in FIG. 13C, the push button 16a of the manipulating portion 16 is released, thereby closing the grasping forceps 7 to grasp the distal end of the atrial appendage A, the manipulating portion 16 is moved backward, and the handle 17 is moved forward, thereby making the loop 14 advance while the atrial appendage A is being pulled.

In this state, if the atrial appendage A is damaged, as shown in FIG. 13D, the handle 18 is moved forward to tighten the loop 14, thereby compressing the atrial appendage A to occlude the internal space B, and making it possible to stop a blood flow into the atrial appendage A. In this way, the atrial-appendage ligation treatment tool 1 and the grasping forceps 7 are moved in the same straight line and are attached so as to allow relative movement, thereby making it possible to improve the ease of manipulation.

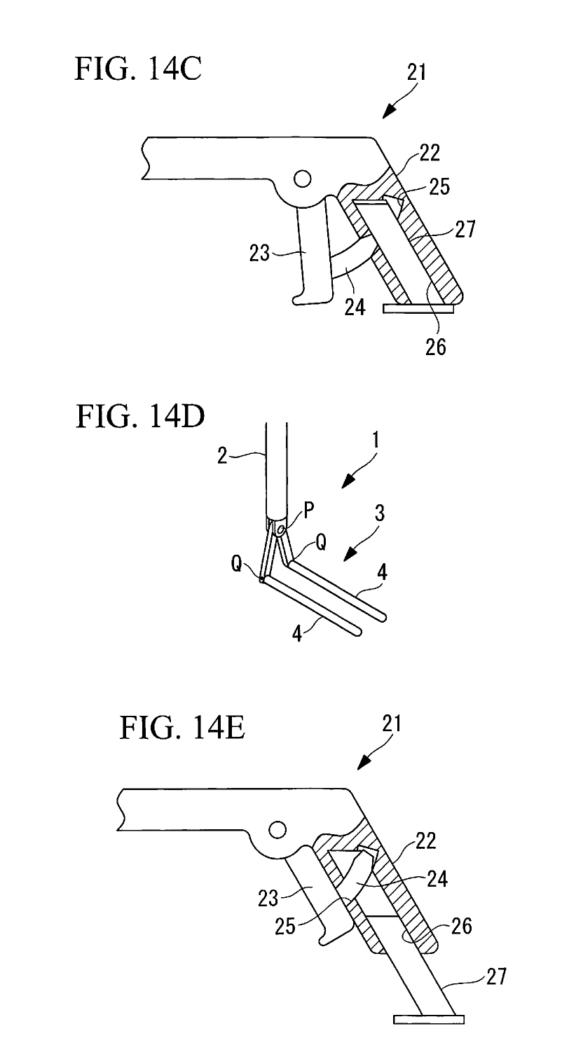

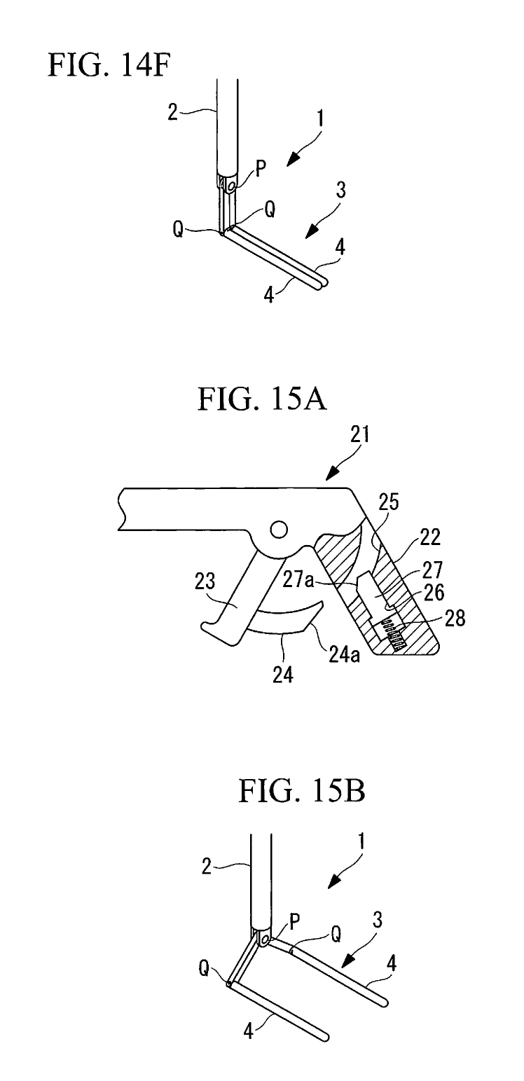

Furthermore, in this embodiment, the space between the two contact parts 4, which constitute the pressing portion 3, is changed between two states, i.e., a state in which the space therebetween is widened and a state in which the space therebetween is narrowed; however, instead of this, as shown in FIGS. 14A to 14F, the space between the contact parts 4 may be changed among three steps.

In the example shown in FIGS. 14A to 14F, a manipulating portion 21 for changing the space between the contact parts 4 is provided with a grasping section 22 and a lever 23 that is provided so as to be rotatable with respect to the grasping section 22. The lever 23 is provided with an arc-like projection 24 that projects toward the grasping section 22. Furthermore, the grasping section 22 is provided with a tunnel section 25 that accommodates the projection 24 when the lever 23 is rotated toward the grasping section 22, an accommodating hole 26 that is provided so as to intersect the tunnel section 25, and a stopper member (stopper) 27 that is movably inserted into the accommodating hole 26.

As shown in FIG. 14A, in a state in which the lever 23 is not pulled, the space between the contact parts 4 is widened to the maximum, as shown in FIG. 14B. In this state, the space between the contact parts 4 is widened sufficiently greater than the thickness dimension of the atrial appendage A, thus making it possible to easily insert the atrial appendage A between the two contact parts 4.

Then, from this state, the lever 23 is pulled to bring it close to the grasping section 22, thereby narrowing the space between the contact parts 4, and, as shown in FIG. 14C, when the distal end of the projection 24 is inserted into the tunnel section 25 and abuts against the stopper member 27, the space between the contact parts 4 is slightly widened, as shown in FIG. 14D. In this state, the space between the contact parts 4 is a space suitable for sandwiching the atrial appendage A disposed between the contact parts 4, in the thickness direction, for tying up the outer surface of the atrial appendage A, and for functioning as the pressing portion 3, which exposes the base of the atrial appendage A in order to loop the ligation loop 8 around the base of the atrial appendage A.

Then, when it becomes necessary to compress the atrial appendage A to occlude the internal space B, thus stopping the flow of blood into the atrial appendage A, as shown in FIG. 14E, the stopper member 27 inserted into the accommodating hole 26 is moved so as to be withdrawn from the accommodating hole 26. Accordingly, the tunnel section 25 occluded by the stopper member 27 is released, thus enabling the operator to further pull the lever 23, and the lever 23 is rotated in the direction in which it is brought even closer to the grasping section 22, thereby making it possible to bring the two contact parts 4 closest to each other, as shown in FIG. 14F, and to compress the atrial appendage A in the thickness direction to occlude the internal space B.

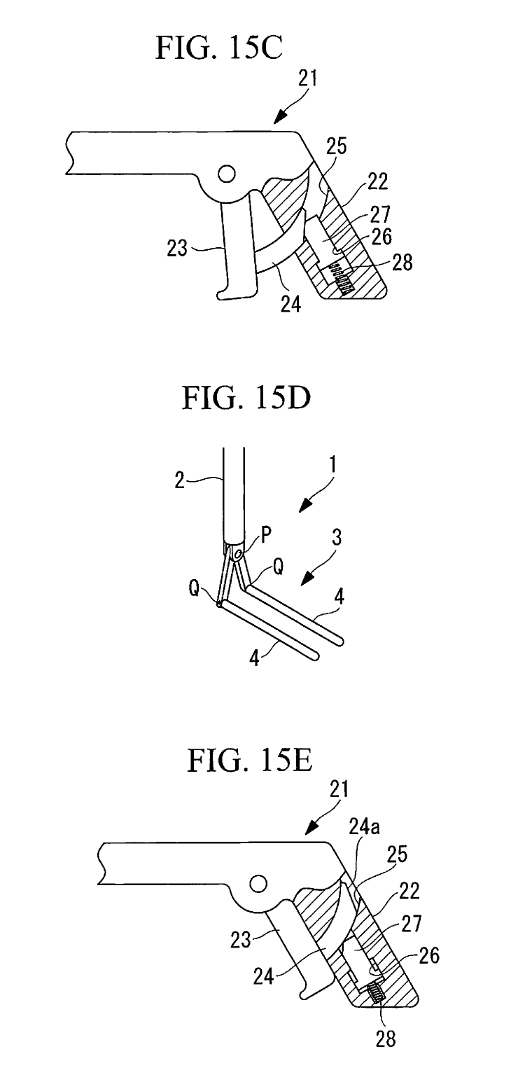

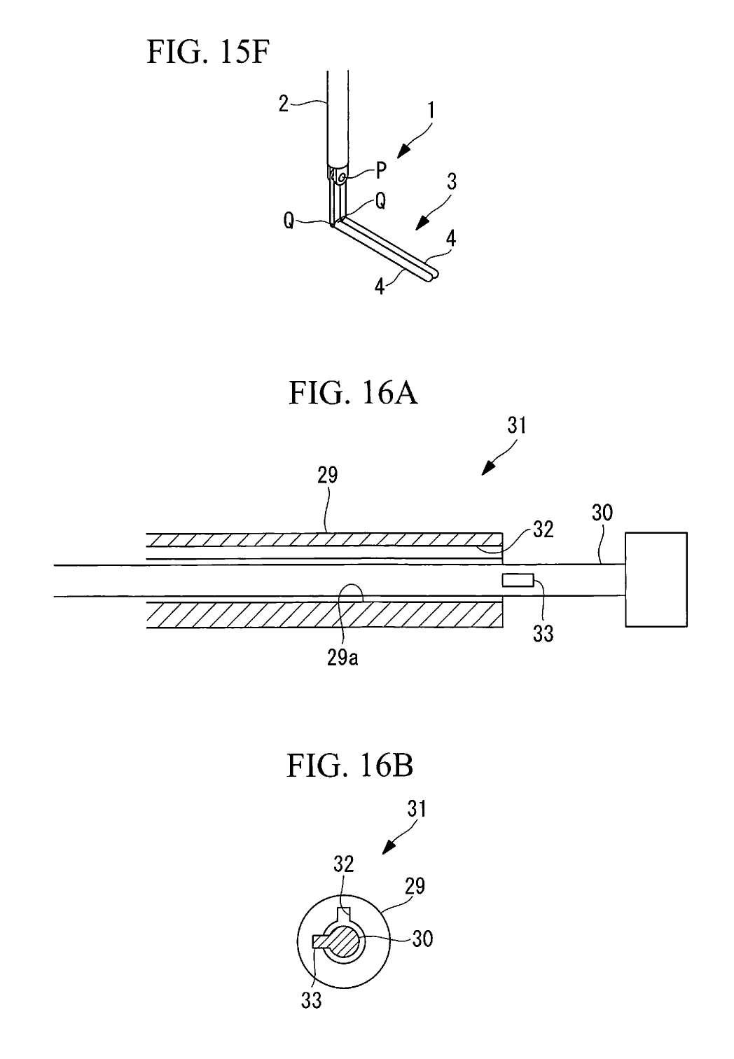

Furthermore, instead of manually withdrawing the stopper member 27, as shown in FIGS. 15A to 15F, the stopper member 27 may be made to project toward the tunnel section 25 by a biasing member 28, such as a spring. In this case, inclined faces 24a and 27a are provided at the distal end of the projection 24 and at the distal end of the stopper member 27, respectively.

The lever 23 is rotated from a state in which the space between the contact parts 4 is widened to the maximum, as shown in FIGS. 15A and 15B, and, when the inclined face 24a of the projection 24 abuts against the inclined face 27a of the stopper member 27, as shown in FIG. 15C, the rotation of the lever 23 is temporarily stopped. In this state, as shown in FIG. 15D, the contact parts 4 are in a state suitable for functioning as the pressing portion 3. Then, when it becomes necessary to stop the flow of blood into the atrial appendage A, an external force is applied so as to bring the lever 23 even closer to the grasping section 22, thereby causing the inclined face 24a of the projection 24 to resist a biasing force of the biasing member 28 to push the stopper member 27 out of the tunnel section 25, as shown in FIG. 15E, and the lever 23 is further rotated, thereby making it possible to bring the two contact parts 4 closest to each other, as shown in FIG. 15F, and to compress the atrial appendage A in the thickness direction to occlude the internal space B.

Furthermore, instead of the manipulating portion 21, in which the lever 23 is rotated, it is possible to adopt a manipulating portion 31 in which a shaft 30 is inserted into and withdrawn from an external cylinder 29, thereby opening and closing the contact parts 4. In this case, as shown in FIGS. 16A and 16B, a key groove 32 is formed on the inner surface of a through-hole 29a in the external cylinder 29, and a projection 33 is provided on the outer surface of the shaft 30, and thus, when the projection 33 does not match the key groove 32, the projection 33 abuts against an end portion of the external cylinder 29, thus stopping the insertion of the shaft 30.

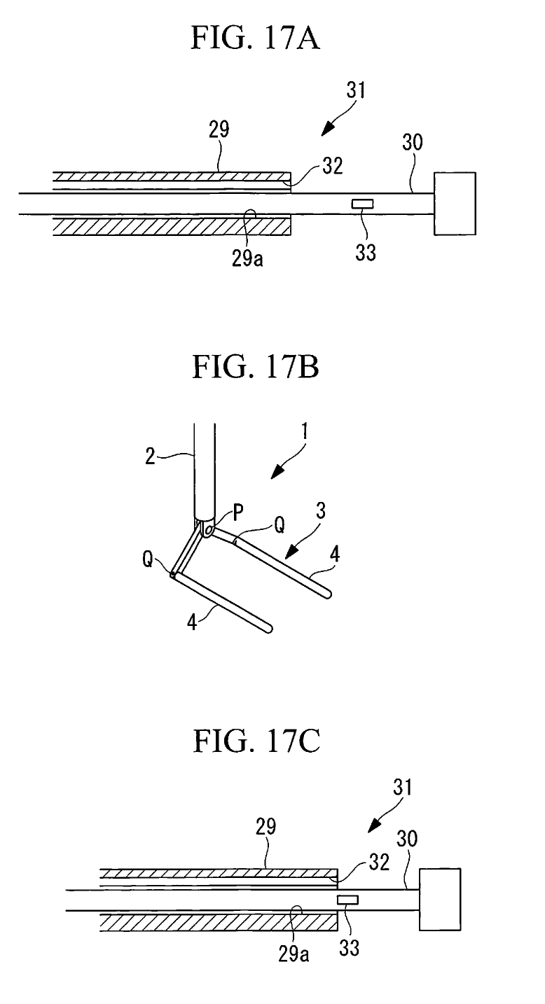

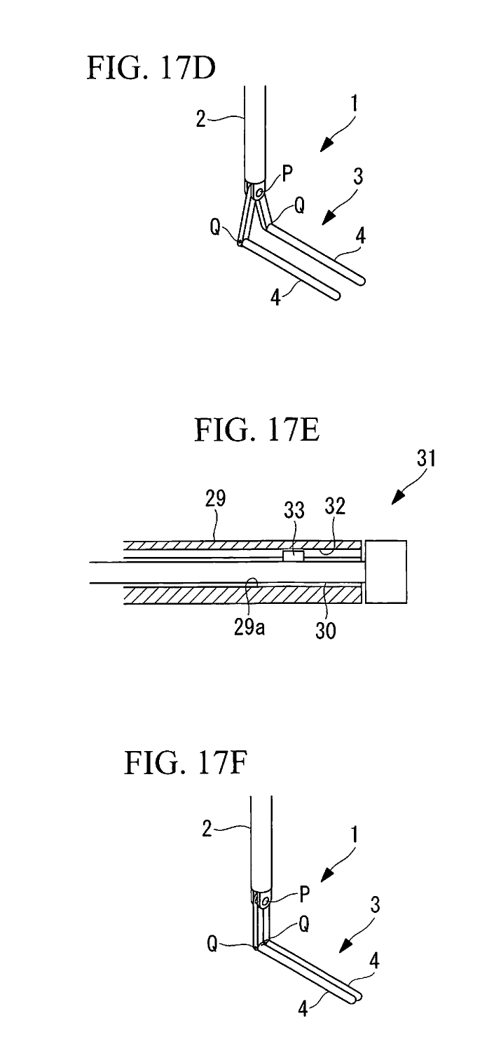

Therefore, when the projection 33 is located away from the end portion of the external cylinder 29, as shown in FIG. 17A, the space between the two contact parts 4 is widened, as shown in FIG. 17B. When the projection 33 abuts against the end portion of the external cylinder 29, as shown in FIG. 17C, the space between the contact parts 4 is narrowed to an intermediate size, as shown in FIG. 17D. Then, when it becomes necessary to stop the flow of blood into the atrial appendage A, the shaft 30 is rotated about the axis to make the phase of the projection 33 match that of the key groove 32, thereby making it possible to insert the projection 33 into the key groove 32 and to further push the shaft 30, as shown in FIG. 17E, and making it possible to bring the two contact parts 4 closest to each other, as shown in FIG. 17F, and to compress the atrial appendage A in the thickness direction to occlude the internal space B.

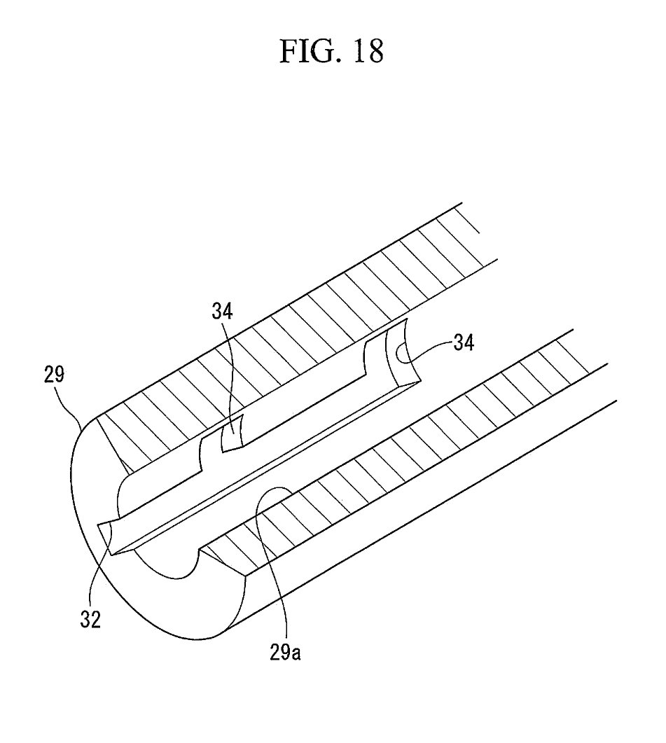

Furthermore, as shown in FIG. 18, circumferential grooves (maintaining mechanism) 34 that extend from the key groove 32 in the circumferential direction are provided, and, when the shaft 30 is inserted into the external cylinder 29 until the projection 33 matches, in position, either of the circumferential grooves 34, the shaft 30 is rotated about the axis to insert the projection 33 into the corresponding circumferential groove 34, thereby making it possible to maintain the space between the contact parts 4 at the corresponding position. Accordingly, the operator does not need to maintain the positional relationship between the shaft 30 and the external cylinder 29 for a long time, thus making it possible to facilitate treatment.

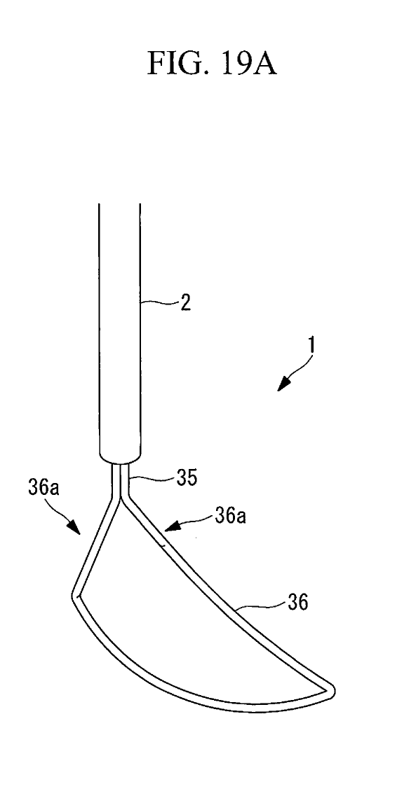

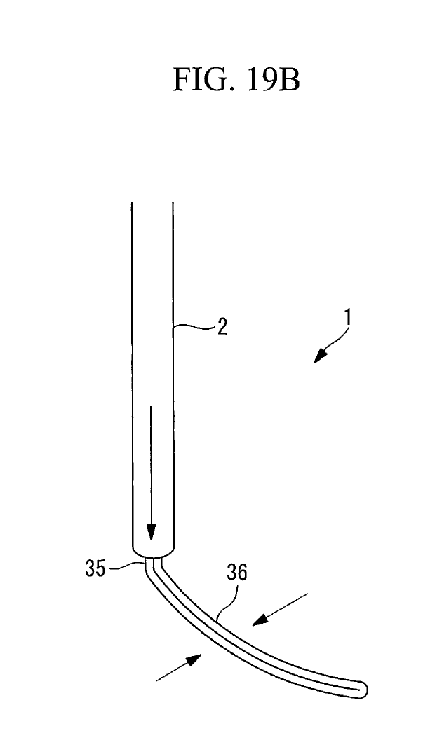

Furthermore, in the examples shown in FIGS. 12A and 12B and in FIGS. 13A to 13D, the loop 14, which is formed of the threadlike wire 13, is used to occlude the internal space B in the atrial appendage A; however, instead of this, as shown in FIGS. 19A and 19B, a loop 36 that is formed of a thick wire 35 having higher stiffness than the threadlike wire 13 may be used to occlude the internal space B in the atrial appendage A. In this case, as shown in FIG. 19A, it is possible to easily maintain an open shape and to make the loop 36 advance to the base of the atrial appendage A by itself, in the same way as in the atrial-appendage ligation treatment tool 1, which has the rod-like contact parts 4, shown in FIG. 1 etc.

Two ends of the wire 35, which forms the loop 36 by being folded back outside the distal-end opening of the through-hole in the shaft 2, are penetrated through the through-hole to extend toward the base end of the shaft 2. Furthermore, the loop 36 is provided with inclined sections 36a that extend from the distal-end opening of the through-hole in the shaft 2, in directions in which they diverge from each other, and, when the wire 35 is withdrawn into the through-hole in the shaft 2, the inclined sections 36a are gradually brought close to each other.

Then, when it becomes necessary to stop the flow of blood into the atrial appendage A, the shaft 2 is pushed to withdraw the two ends of the wire 35 into the shaft 2, thereby making it possible to deform the loop 36 so as to be folded and to sandwich the atrial appendage A disposed therein, in the thickness direction. Because the atrial appendage A is sandwiched by using the loop 36, whose distal ends are connected, the sandwiching force is strong, and thus the internal space B in the atrial appendage A can be more reliably occluded, without yielding to the hardness of the atrial appendage A.

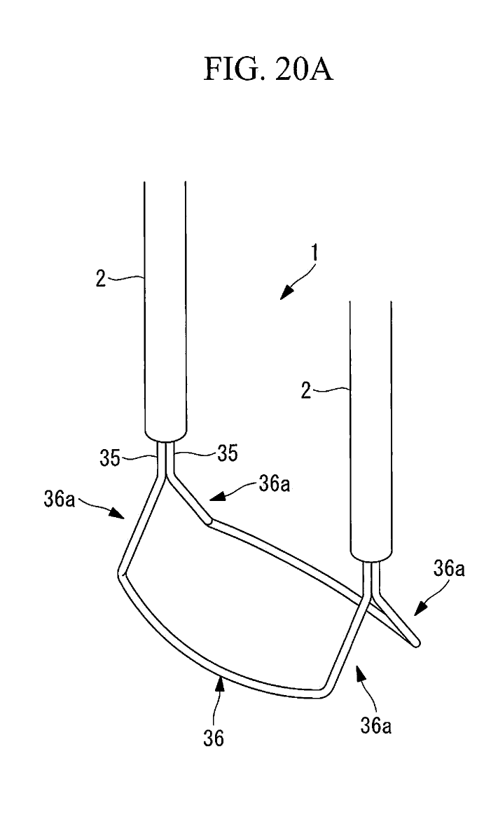

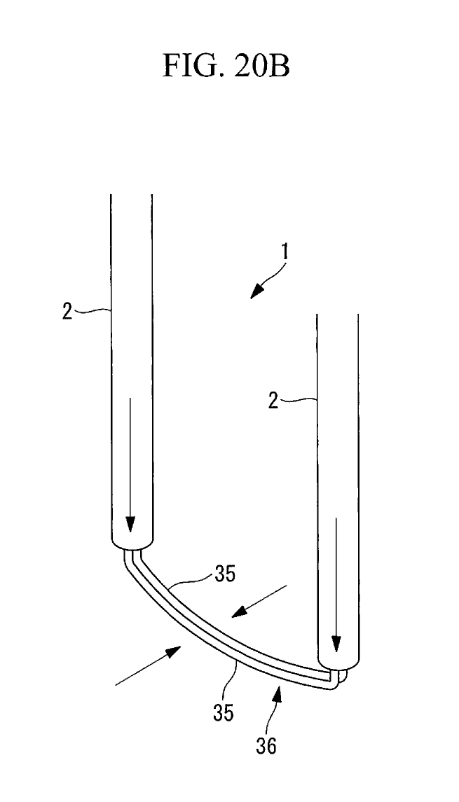

Furthermore, instead of supporting the loop 36 with the one shaft 2, as shown in FIGS. 20A and 20B, it is possible to adopt an atrial-appendage ligation treatment tool 1 that has a configuration in which two wires 35 are supported between two shafts 2, and both ends of the respective wires 35 are withdrawn into the shafts 2, thereby narrowing the space between the wires 35. Inclined sections 36a are provided at the outsides of the distal-end openings of the through-holes in the two shafts 2, thereby making it possible to evenly narrow the space between the two wires 35, which are disposed between the shafts 2, and to evenly compress the atrial appendage A in the width direction to occlude the internal space B more reliably.

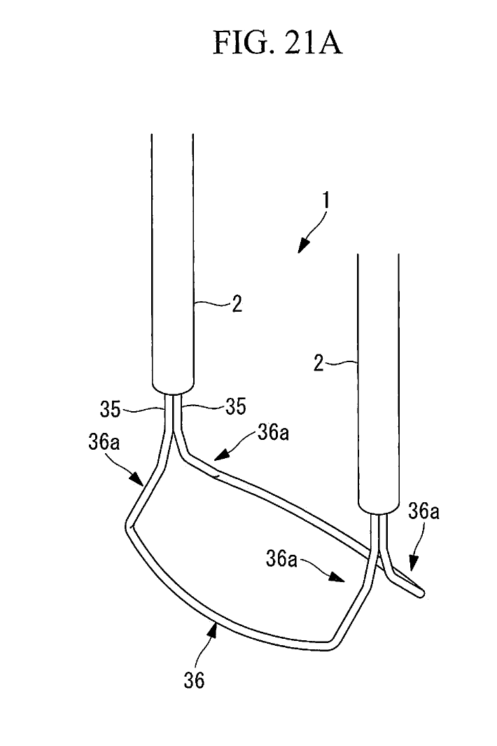

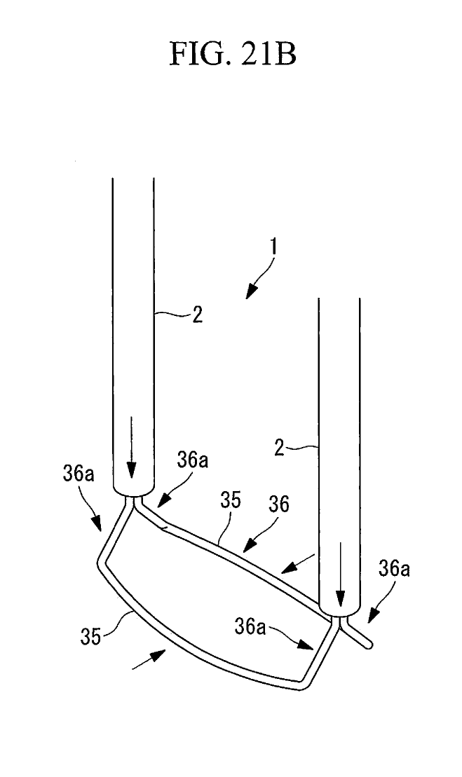

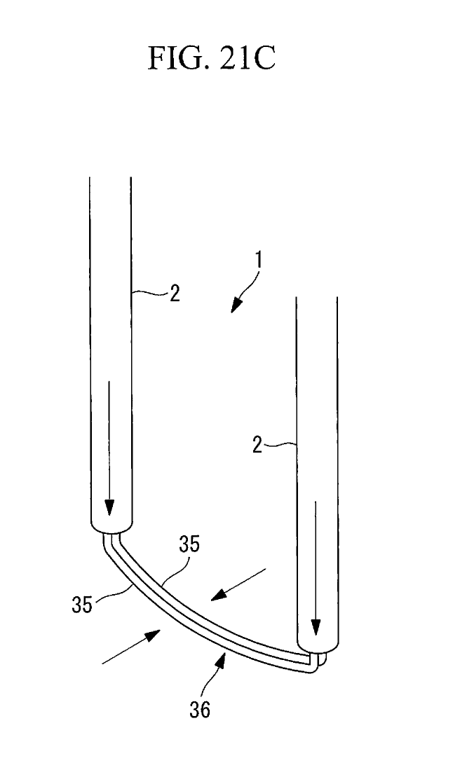

Furthermore, the inclined sections 36a are each provided with two-step inclination angles, as shown in FIG. 21A, and, from the state in which the space between the wires 35, which are disposed between the shafts 2, is widened to the maximum, as shown in FIG. 21A, the wires 35 are pulled while the shafts 2 are being pushed forward at the base ends of the shafts 2, thereby allowing a change to a state in which the space between the wires 35 is narrowed, as shown in FIG. 21B, and in which they can easily exhibit their function as the pressing portion 3. By further pulling the wires 35 from this state, as shown in FIG. 21C, the space between the wires 35 is narrowed to the minimum, thus allowing a change, in a step-like manner, to a state in which the internal space B in the atrial appendage A is occluded.

A procedure for ligating the atrial appendage A using the atrial-appendage ligation treatment tool 1 of this embodiment will be described below.

First, the distal end of the atrial appendage A is grasped by the grasping forceps 7, which are introduced into the cardiac sac from outside the body through the sheath 5 penetrated to the inside of the cardiac sac from a lower portion of the ensiform cartilage, and the atrial appendage A is stretched (grasping step). Next, the ligation loop 8, which is introduced to the cardiac sac through the sheath 5, is looped around the atrial appendage A being stretched, and the ligation loop 8 is disposed at the base of the atrial appendage A (loop disposing step). Then, the wire forming the ligation loop 8 is pulled outside the body, thereby tightening the ligation loop 8 to ligate the atrial appendage A (ligation step).

In this state, while moving the atrial appendage A by manipulating the grasping forceps 7, the state of ligation of the atrial appendage A using the ligation loop 8 is confirmed by using an endoscope or through transesophageal ultrasound observation (confirmation step).

After the ligation state is confirmed, the atrial appendage A grasped by the grasping forceps 7 is released from the grasping forceps 7, and the grasping forceps 7 are extracted to the outside of the body. Furthermore, the loop shaft 9, which has supported the ligation loop 8, is also extracted to the outside of the body (extraction step). Then, scissor forceps (not shown) are introduced to the cardiac sac through the sheath 5 to cut excess wire of the ligation loop 8 (cutting step).

Here, in the above-described grasping step and ligation step, tissue of the atrial appendage A may be damaged, and, in such cases, the internal space B in the atrial appendage A is occluded by using the atrial-appendage ligation treatment tool 1 of this embodiment. Specifically, the atrial-appendage ligation treatment tool 1 is manipulated outside the body such that the atrial appendage A is disposed between the contact parts 4 provided at the distal end of the atrial-appendage ligation treatment tool 1, which is introduced through the sheath 5. If the atrial-appendage tissue is damaged, the atrial-appendage ligation treatment tool 1 is manipulated to narrow the space between the contact parts 4, thereby compressing the atrial appendage A from the outer surface thereof to occlude the internal space B. The same procedure is applied when the loop 14 or 36 is used for occluding the internal space B.

Note that the contact parts 4 of the atrial-appendage ligation treatment tool 1 may be positioned prior to the grasping step or the ligation step, thereby making it possible to immediately compress the atrial appendage A to occlude the internal space B when the tissue of the atrial appendage A is damaged.

Furthermore, the confirmation step may be performed after the extraction step or the cutting step. Furthermore, the extraction step may be performed after the cutting step.

Furthermore, as shown in FIGS. 14A to 18 and FIGS. 21A to 21C, when the space between the contact parts 4 or the size of the loop 36 is changed in a step-like manner, it is necessary to dispose the atrial appendage A between the contact parts 4 of the atrial-appendage ligation treatment tool 1 or in the loop 36 thereof immediately after the grasping step and to expose the base of the atrial appendage A by using, as the pressing portion 3, the contact parts 4 or the loop 36, with the space therebetween or therein being narrowed halfway, prior to the ligation step. Then, if the tissue of the atrial appendage A is damaged in any step, the space between the contact parts 4 or the size of the loop 36 may be immediately narrowed to the minimum to occlude the internal space B in the atrial appendage A.

The above-described embodiment leads to the following inventions.

According to one aspect, the present invention provides an atrial-appendage ligation treatment tool that is used, together with an atrial-appendage ligation tool having a ligation part, to perform atrial-appendage ligation treatment, the atrial-appendage ligation treatment tool including: a compressing portion that is introduced, separately from the ligation part, to a cardiac sac through a sheath penetrated through a pericardium and that compresses an atrial appendage from outside to occlude an internal space therein; and a shaft that is disposed in the sheath in an inserted manner and that supports, at a distal end thereof, the compressing portion.

According to this aspect, the atrial-appendage ligation treatment tool is introduced to the cardiac sac through the sheath penetrated through the pericardium, the shaft is pushed in the longitudinal direction at the base end of the sheath, the compressing portion, which is provided at the distal end of the shaft, is made to advance in the cardiac sac toward the atrial appendage, and a distal end portion thereof is disposed in the vicinity of the atrial appendage, for example, in the vicinity of the base of the atrial appendage. At this time, the compressing portion may be positioned by using an observation unit, such as an endoscope, that is separately inserted or an X-ray transparent image. Because the sheath is inserted into the cardiac sac from the ensiform cartilage side, the atrial-appendage ligation treatment tool can be brought close to the atrial appendage from the distal end thereof.

Then, when the distal end of the atrial appendage is grasped and pulled by the grasping forceps to apply the ligation part to the atrial appendage or when the ligation treatment is performed by using the ligation part, if a wrong level of force is exerted in manipulating the grasping forceps or the ligation part, the atrial-appendage tissue may be damaged in some cases. In such cases, the compressing portion of the atrial-appendage ligation treatment tool according to this aspect is used to compress the atrial appendage from outside at a position that is closer to the base than a ligation position, at which ligation is performed using the ligation part, is, or at a position that is closer to the base than a grasping position, at which grasping is performed using the grasping forceps, is, thereby making it possible to occlude the internal space in the atrial appendage and to minimize the effect of the damage.

In the above-described aspect, the compressing portion may be provided with two rod-like contact parts that can be disposed at a position where the atrial appendage is to be sandwiched in the thickness direction, over the entire length of the atrial appendage in the width direction; and a space between the contact parts may be changed.

By doing so, at the time of insertion through the sheath, the space between the two contact parts is narrowed to make the compressing portion compact, thus making it possible to improve the ease of insertion. After introduction to the cardiac sac, the space between the two contact parts is widened, thus making it possible to improve the ease of insertion of the atrial appendage therebetween. Furthermore, with the two contact parts being disposed on both sides of the atrial appendage, the space between the two contact parts is narrowed, thus making it possible to sandwich the atrial appendage in the thickness direction, to compress it from outside, and to occlude the internal space in the atrial appendage.

Furthermore, in the above-described aspect, it is possible to further include a biasing part that exerts a bias such that the contact parts are aligned with the shaft in the sheath and, when released from an inside of the sheath, extend in a direction intersecting the longitudinal axis of the shaft.

By doing so, when the atrial-appendage ligation treatment tool is introduced to the cardiac sac through the sheath penetrated through the pericardium, the compressing portion, which is aligned with the shaft in the sheath, is biased by the biasing part when released from the sheath in the cardiac sac, thus extending in a direction intersecting the longitudinal axis of the shaft. Accordingly, it is possible to dispose the contact parts across the atrial appendage in the width direction and to occlude the internal space in the atrial appendage to stop the blood flow into the atrial appendage.

Furthermore, in the above-described aspect, the biasing part may be configured such that the shaft is formed of an elastic material and has a pre-bent section.

By doing so, without providing a joint mechanism, it is possible to change the angle of the compressing portion from a state where the compressing portion is aligned with the shaft to a state where the compressing portion is inclined with respect to the shaft. Accordingly, it is possible to compactly configure the shaft and the compressing portion and to improve the ease of insertion through the sheath.

Furthermore, in the above-described aspect, the contact parts may be rotatably supported at the distal end of the shaft by a joint.

Furthermore, in the above-described aspect, one of the contact parts may be provided with a magnetic body, and the other thereof may be provided with a magnet.

By doing so, without providing a driving mechanism for bringing the contact parts close to each other, with the contact parts being disposed on both sides of the atrial appendage in the thickness direction, it is possible to bring the two contact parts close to each other due to the magnetic force between the magnet and the magnetic body, and to firmly sandwich the atrial appendage to maintain the internal space in an occluded state.

Furthermore, in the above-described aspect, it is possible to further include two or more holding parts that can be disposed around the atrial appendage, in which the compressing portion may be a loop-like wire that is releasably held by the holding parts and that can be tightened.

By doing so, after the shaft is manipulated to insert the atrial appendage into the loop-like wire so as to dispose the two or more holding parts, which hold the loop-like wire, around the atrial appendage, the loop-like wire is released from the holding parts and is tightened, thereby making it possible to tighten the atrial appendage to occlude the internal space.

Furthermore, in the above-described aspect, the space between the two contact parts of the compressing portion may be changed in a step-like manner.

By doing so, the space between the contact parts is largely widened, thus improving the ease of insertion of the atrial appendage; the space therebetween is narrowed halfway and is pressed against the base of the atrial appendage, thus making it possible to expose the base of the atrial appendage for ligation; and the space therebetween is narrowed to the minimum, thus making it possible to occlude the internal space in the atrial appendage.

Furthermore, in the above-described aspect, it is possible to further include a manipulating portion that is manipulated, at a base end of the shaft, to change the space between the two contact parts, in which the manipulating portion may be provided with a stopper that releasably prohibits manipulation of the manipulating portion while the space between the contact parts is being changed.

By doing so, manipulation of the same manipulating portion is prohibited by the stopper, thus making it possible to temporarily stop the manipulation in a state in which the space between the contact parts is narrowed halfway, and the stopper is released, thus making it possible to narrow the space between the contact parts to the minimum. Accordingly, with the same manipulating portion, the space between the contact parts can be easily changed in a step-like manner.

Furthermore, in the above-described aspect, it is possible to further include a maintaining mechanism that can maintain the space between the contact parts of the compressing portion, in each step.

By doing so, an operator can eliminate the effort required to maintain the space between the contact parts, thus easily performing treatment.

Furthermore, according to another aspect, the present invention provides an atrial-appendage ligation system including: grasping forceps that grasp an atrial appendage; and one of the above-described atrial-appendage ligation treatment tools.

In the above-described aspect, the grasping forceps and the compressing portion may be provided so as to be relatively movable in the longitudinal direction.

### REFERENCE SIGNS LIST

A atrial appendage
B internal space
P joint
1 atrial-appendage ligation treatment tool
2 shaft
3 pressing portion (compressing portion)
4 contact parts
5 sheath
7 grasping forceps
8 ligation loop (ligation part)
10 magnetic body
11 electromagnet (magnet)
14 loop (compressing portion)
15 holding part
20 atrial-appendage ligation system

1. An atrial-appendage ligation treatment tool that is used, together with an atrial-appendage ligation tool having a ligation part, to perform atrial-appendage ligation treatment, the atrial-appendage ligation treatment tool comprising:
a compressing portion that is introduced, separately from the ligation part, to a cardiac sac through a sheath penetrated through a pericardium and that compresses an atrial appendage from outside to occlude an internal space therein; and
a shaft that is disposed in the sheath in an inserted manner and that supports, at a distal end thereof, the compressing portion.

2. An atrial-appendage ligation treatment tool according to claim 1,
wherein the compressing portion is provided with two rod-like contact parts that can be disposed at a position where the atrial appendage is to be sandwiched in the thickness direction, over the entire length of the atrial appendage in the width direction; and
a space between the contact parts can be changed.

3. An atrial-appendage ligation treatment tool according to claim 2, further comprising a biasing part that exerts a bias such that the contact parts are aligned with the shaft in the sheath and, when released from an inside of the sheath, extend in a direction intersecting the longitudinal axis of the shaft.

4. An atrial-appendage ligation treatment tool according to claim 3, wherein the biasing part is configured such that the shaft is formed of an elastic material and has a pre-bent section.

5. An atrial-appendage ligation treatment tool according to claim 3, wherein the contact parts are rotatably supported at the distal end of the shaft by a joint.

6. An atrial-appendage ligation treatment tool according to claim 2, wherein one of the contact parts is provided with a magnetic body, and the other thereof is provided with a magnet.

7. An atrial-appendage ligation treatment tool according to claim 1, further comprising two or more holding parts that can be disposed around the atrial appendage,
wherein the compressing portion is a loop-like wire that is releasably held by the holding parts and that can be tightened.

8. An atrial-appendage ligation treatment tool according to claim 2, wherein the space between the two contact parts of the compressing portion can be changed in a step-like manner.

9. An atrial-appendage ligation treatment tool according to claim 8, further comprising a manipulating portion that is manipulated, at a base end of the shaft, to change the space between the two contact parts,
wherein the manipulating portion is provided with a stopper that releasably prohibits manipulation of the manipulating portion while the space between the contact parts is being changed.

10. An atrial-appendage ligation treatment tool according to claim 8, further comprising a maintaining mechanism that can maintain the space between the contact parts of the compressing portion, in each step.

11. An atrial-appendage ligation system comprising:
grasping forceps that grasp an atrial appendage; and
an atrial-appendage ligation treatment tool according to claim 1.

12. An atrial-appendage ligation system according to claim 11, wherein the grasping forceps and the compressing portion are provided so as to be relatively movable in the longitudinal direction.
